(19) United States
(12) Patent Application Publication
(10) Pub. No.: US 2021/0000453 A1
(43) Pub. Date: Jan. 7, 2021

(54) Acquisition of Samples for Evaluating Bacterial Demographics

(71) Applicants: Sameer Sonkusale, Lincoln, MA (US); Hojatollah Rexaei Nejad, Newton, MA (US); Trustees of Tufts College, Medford, MA (US)

(72) Inventors: Sameer Sonkusale, Lincoln, MA (US); Hojatollah Rexaei Nejad, Newton, MA (US)

(21) Appl. No.: 16/979,957

(22) PCT Filed: Mar. 12, 2019
(86) PCT No.: PCT/US19/21832

§ 371 Date: Sep. 11, 2020

Related U.S. Application Data
(60) Provisional application No. 62/641,749, filed on Mar. 12, 2018.

(51) Int. Cl. A61B 10/00

(52) U.S. Cl. CPC A61B 10/0045; A61B 5/073; A61B 2010/0061

(57) ABSTRACT

A gut rover traverses the guy and collects samples of the microbiome in a way that permits correlation of samples with particular locations from which they were sampled.

### RELATED APPLICATIONS

This application claims the benefit of the Mar. 12, 2018 priority date of U.S. Provisional Application 62/641,749, the contents of which are herein incorporated by reference.

### STATEMENT OF GOVERNMENT RIGHTS

This invention was made with government support under grant N00014-16-1-2550 awarded by the United States Navy. The government has certain rights in the invention.

### FIELD OF INVENTION

The invention pertains to bacterial demographics, and in particular, to identifying the spatial distribution of bacterial species within a region that is not easily accessible.

### BACKGROUND

The gut microbiome has profound effects on the development and maintenance of the immune system in both animal models and in humans. A growing body of evidence has implicated the human gut microbiome in a range of disorders, including obesity, inflammatory-bowel diseases, cancer, and cardiovascular disease. The gut microbiome represents 100 trillion bacteria, most of which belong to a thousand or so bacterial species. Studies examining this bacterial content have shown wide variations in which species are present between individuals.

The gut, however, is difficult to access. One cannot simply swab a particular portion of the gut to obtain a sample of the bacterial population at that portion. Instead, the usual procedure is to analyze fecal matter. However, one cannot tell from inspecting fecal matter exactly which area of large or small intestine the bacterial species colonize and how they interact with one another and with the host.

To gain new insights into the role of gut microbiome, it is useful to sample the microbiome at different locations in the gut to obtain a spatial distribution profile. Such studies are currently not possible with the fecal matter analysis.

### SUMMARY

This invention relates to an orally-administered gut rover that travels through the gut and obtains samples of the microbiome in such a way that the location from which the sample was taken can be identified. As used herein, the “gut” includes both the large intestine and the small intestine.

In one aspect, the invention features a gut rover that is configured to traverse a gut. Such a gut rover includes a sampler for obtaining samples of the microbiome at selected locations within the gastrointestinal system.

A variety of samplers are available. Among these are an osmotic sampler in which an osmotic pressure differential across a membrane drives sampling. Among these are embodiments in which the sampler is configured to halt sampling upon having collected a pre-defined volume.

In some embodiments, the sampler comprises a brine reservoir, a semi-permeable membrane, and a collection chamber that is in fluid communication with an inlet through which fluid within the gut can enter the gut rover. In these embodiments, the semi-permeable membrane separates the brine reservoir from the collection chamber. Among these are embodiments in which the brine reservoir has a volume that expands during sampling.

In other embodiments, the sampler comprises an oil reservoir, a back channel, and an elastic membrane. In these embodiments, the elastic membrane separates a brine reservoir from the oil reservoir and, when deformed, increases a level of oil from the oil reservoir in the back channel.

In other embodiments, the sampler comprises a thread and nodes along the thread, wherein the thread has an end exposed to fluid within the gut.

In yet other embodiments, the sampler comprises a material that changes shape in response to a trigger. Among these are embodiments in which the sampler comprises a material that transitions between hydrophobic and hydrophilic states in response to an energy input.

In yet other embodiments, the sampler comprises tentacles that deform between an open state, in which the tentacles are exposed to fluid in the gut, and a closed state, in which the tentacles entrap samples from the fluid.

Also among the embodiments are those in which the sampler comprises a heater that is selectively activated by a remote trigger.

Other embodiments feature a pump that is connected to an inlet of the gut rover.

In some embodiments, the pump is a peristaltic pump.

In yet other embodiments, the sampler comprises a screw having threads, wherein pairs of threads confine fluid therebetween. As the screw rotates, fluid confined between pairs of threads moves along an axis of the screw.

In still other embodiments, the sampler comprises an endless belt that extends between first and second pulleys, wherein the endless belt follows a path that exposes the belt to gut fluid.

Still other embodiments feature a shield that prevents fluid from contacting the sampler, wherein the shield is configured to dissolve upon occurrence of a condition indicative of entry into region from which samples are to be acquired.

In some embodiments, the apparatus comprise metal that responds to magnetic field or a magnet so that it can be tracked using an external magnetic reader. This permits its location in the gut to be determined.

A useful way to track the rover is to surround the patient with an array of preferably tri-axial magnetometers. Each magnetometer will measure a magnetic field vector resulting from the magnet within the rover. This results in a system of equations in which the coordinates of the rover are the unknowns. Once the system of equations is defined, it can be solved, typically iteratively or numerically, to identify the correct coordinates of the magnet, and hence, the rover. In a typical case, the cage would have eight magnetometers.

One approach is to measure the magnetic field generated by the magnet at selected locations within the cage during a calibration step and to then infer the location within the patient based on these calibrated fields. A useful algorithm for solving the system of equations is the Levenberg-Marquardt nonlinear least squares optimization algorithm. In solving the equations, the Earth's magnetic field and any ambient field are considered, thus avoiding the need to provide shielding.

In other embodiments, the pill is configured to be tracked using ultrasound, MRI, or optical imaging. These all permit identifying the exact location in the gut.

Some embodiments of the manufacture passively surf the peristaltic waves on its way through the stomach and the gut. However, other embodiments use the magnet or metal inside the pill for external control over the pill movement and location inside the gut. In some cases, an external magnet holds the pill in place inside the gut to enable longer duration of sampling from that particular region of the gut.

Once sampling is complete, the pill is expelled with feces and recovered. The contents can then be extracted for further downstream analysis. To more easily identify the rover 10 in the feces, it is useful to provide a fluorescent dye or to have the outer surface be of a particularly conspicuous color, and in particular, to avoid brown.

In some embodiments, the manufacture includes a battery or super capacitor and electronic circuitry to provide sampling, for example by actuating pumps, motors, and similar devices.

Embodiments further include those in which sampling is carried out with no external power source. These devices rely on osmotic pumps and capillary pumps.

Also among the embodiments are those in which sampling requires an energy source. In some embodiments, the energy source harvests mechanical energy from gut movement. In others, the energy source features a battery. Among these are those in which the gastric fluid itself serves as an electrolyte medium for such a battery.

Further embodiments include those in which the pills have been encased in an enteric coating to protect the pill as it passes through the stomach. The coating is configured to dissolve only in the gut, where the pill starts sampling.

The timing of sampling can be controlled in other ways. For example, it is possible to delay the start of sampling in the gut using a hydrogel or polymer coating at the inlet of the pill. The composition and thickness of such a coating dictates its dissolution rate, and hence the start of the sampling procedure. On complete dissolution of this coating, the sampling process can be initiated. Delaying the sampling process provides a way to control which areas of the gut are to be sampled. This is particularly useful since the pill's volume is finite, and therefore the pill can only collect a finite sample volume.

Another way to control the sampling starting time is to actively do so using an external trigger mechanism. Examples include the use of a reed switch that responds to magnetic field. A reed switch that causes an inlet valve to open can be actuated through thermal, electrochemical, electrical, magnetic, or chemical triggers. An alternative way to trigger the sampling procedure is to have an on-board radio receiver that receives, via a radio signal from an external source, an instruction to begin sampling.

In some embodiments, the manufacture includes a gastro-intestinal positioning system to identify its location within the GI tract. In some embodiments, the gastro-intestinal positioning system includes sensors to which regions of the stomach or the gut it is located in. Such devices make use of the environmental characteristics of different regions as a basis for intra-gastric location. As an example, hydrogen ion concentrations vary considerably be used to easily identify whether the pill is in the upper or lower stomach, duodenum, large or small intestine. Therefore, a sensor that can sense concentrations of ionic species, and in particular hydrogen or hydroxide ions, is particularly useful for intra-gastric location. One can also employ chemical and biological sensors to help identify the location in which the pill is in the gut without the need for any camera. This is particularly useful because illumination levels within the gut tend to be very low.

Depending on the nature of the pumps, different sampling mechanisms are possible. Examples include osmotic sampling, in which an osmotic pressure differential across a membrane drives sampling; capillary sampling, in which natural capillary action in hydrophilic materials, such as textiles or paper, drive sampling; screw-pump sampling, in which a screw driven by an electrical motor drives sampling, and chemical soft actuators, in which sampling is driven by folding of responsive polymers, or through the use of chemical adhesives or glues to trap sampled particles.

In another aspect, the invention features providing a gut rover to a patient, after the gut rover has traversed the patient's gut, recovering the gut rover, and recovering, from the gut rover, microbes from within the gut.

Some practices include tracking the gut rover while the gut rover traverse the gut. Among these practices are those that include observing a magnetic signature from the gut rover and identifying a location of the gut rover based on the magnetic signature. Other practices including tracking via ultrasound and tracking using MRI.

Other practices include controlling sampling by the gut rover while the gut rover is within gut. Among these practices are those in which controlling sampling includes causing a heater within the gut rover to generate heater and those in which wherein controlling sampling comprises turning on a motor within the gut rover.

Yet other practices include reorienting the gut rover while the gut rover is within the gut. Among these are practices that include exposing the gut rover to a magnetic field generated outside the patient.

Still other practices of the invention include causing the gut rover to move while the gut rover is within the gut. Among these practices are those that include causing the gut rover to move comprises exposing the gut rover to a magnetic field generated outside the patient.

There exist a variety of ways to recover microbes from the gut rover. Among these include recovery of fluid that has been trapped behind an oil plug, recovery of a thread that has been impregnated with fluid that has been gathered from the gut through capillary action, and recovery from a fluid having a first concentration of water, wherein the gut has fluid that has a second concentration of water, and wherein the first concentration is less than the second concentration.

### BRIEF DESCRIPTION OF THE DRAWINGS

FIG. 1 shows a gut rover having a sampler;

FIGS. 2-5 shows the sampler of FIG. 1 implemented using shape-shifting tentacles;

FIG. 6 shows a sampler that relies on capillary flow for collection of samples;

FIG. 7 shows a gut rover that relies on osmotic pressure for collection of samples;

FIG. 8 shows an osmotic sampler similar to that shown in FIG. 7 but with a back channel to allow oil to form a plug that stops collection;

FIG. 9 shows the osmotic sampler shown in FIG. 8 with the oil having formed the plug;

FIG. 10 shows a sampler that relies on a peristaltic pump to collect samples;

FIG. 11 shows a sampler that relies on a sticky belt to collect samples; and

FIG. 12 shows a sampler that relies on a screw to collect samples.

### DETAILED DESCRIPTION

FIG. 1 shows a gut rover 10 that is suitable for collecting samples of the microbiome as it traverses the gut 12. A gut rover 10 includes a housing shaped like a capsule or pill so that it can begin its journey along the gut 12 by being swallowed. The housing houses an instrument section 14 and a collecting section 16.

The instrument section 14 houses instrumentation that permits the gut rover 10 to be controlled and guided during its journey along the gut 12. It also permits two-way communication with the gut rover 10.

The instrument section 14 houses a magnet 18 to enable the gut rover 10 to be moved or oriented by application of a magnetic field from outside the body. This permits the gut rover 10 to be propelled without having to rely exclusively on peristalsis for its motion. This magnet 18 also permits the gut rover 10 to be held at a location within the gut 12 for an extended sampling period or to be moved backwards against peristaltic flow to re-sample an upstream section of the gut 12.

The instrument section 14 also includes a number of optional features, including a sensor system 20 that can perform analysis on gut fluid and a communication system 22 with an associated antenna 24 so that the results of such an analysis can be transmitted to an external controller 26. In those cases that rely on a motor for sampling, the communication system 22 provides a way to stop and start the motor.

The collecting section 16 houses a sampler 28 that is exposed to gut fluid so as to sample microbes that characterize the gut's microbiome.

A typical collecting section 16 features one or more inlets 30. Fluid from the gut flows into the inlet 30 so that the sampler 28 is able to collect microbes from its environment. In some embodiments, the inlet 30 permits exposure of the collecting section 16 to gut fluids. The inlet 15 can also be used to insert fluid to prime a sampler 28 within the collecting section 16 prior to having the patient swallow the gut rover 10. After the gut rover 10 has been recovered from the feces, the inlet 30 provides an avenue for pipetting the sample out of the collecting section 16.

Other embodiments also feature an outlet 32 so that gut fluid can flow from the inlet 30 to the outlet 32.

The gut rover 10 in FIG. 1 is shown shortly after having left the stomach 34 and entered the small intestine 36, from which it will eventually traverse the large intestine 38 and be expelled through the anus 40.

In use, a patient swallows the gut rover 10. Natural peristaltic action then propels the gut rover 10 through the gut 12. As the gut rover 10 travels through the gut 12, it acquires samples. Once expelled from the gut 12, the gut rover 10 can be recovered and the samples extracted therefrom. An external controller 26 provides communication with and control over the gut rover 10 as it traverses its path.

In those embodiments that include the sensor system 20, the sensors can be physical or chemical sensors. Examples of chemical sensors include a pH sensor to map the local pH profile of the gut and sensors for various molecules, such as dissolved carbon dioxide, ammonia, pyocyamin, or nicotinamide adenine dinucleotide. Examples of biological sensors include antibody-functionalized sensors for detection of specific microbes and for detection of endotoxins for signs of infection by Clostridium difficile.

In those embodiments with a communication system 22, a suitable communication system 22 is one made from a CMOS integrated circuit with a wireless interface to communicate with entities outside the gut rover 10 and outputs for communicating with electrical devices carried on board the gut rover 10. Typically, an energy source will be required on board to power the communication system 22.

A number of different kinds of samplers 28 can be used within the collecting section 16. Among these are chemical soft actuators, osmotic pumps, capillary pumps, and mechanical pumps, including peristaltic pumps and pumps that drive a sampling belt.

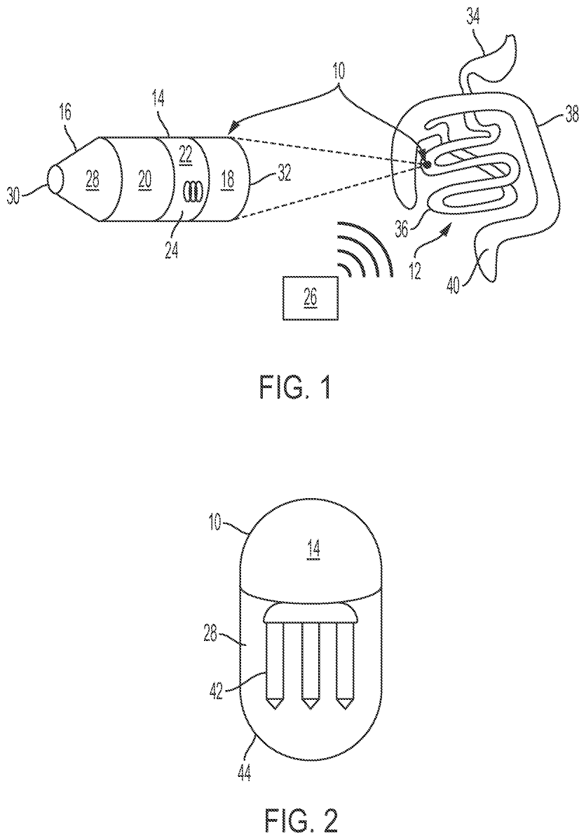

FIG. 2 shows a gut rover 10 in which the sampler 28 is a soft actuator within the collecting section 16. The actuator features tentacles 42 that change shape on cue. In the illustrated embodiment, the tentacles 42 comprise a shape-shifting material. Such a material will promote mechanical bending of the tentacles 42, thus permitting them to grasp, hold, or release.

In FIG. 2, the tentacles 42 remain sheathed within a shield 44. Such a shield 44 is configured to dissolve when the gut rover 10 reaches its target area, thus avoiding premature sampling. A change in the local chemical environment can be used to trigger dissolution of the shield 44. The particular change dictates the material from which the shield 44 will be made. A variety of polymers are known to dissolve in response to particular stimuli.

In the illustrated embodiment, the change in the local chemical environment is a change in pH. As a result, the shield 44 is made of a pH-responsive polymer that dissolves when it encounters the higher pH within the intestine. Such a shield 44 can also be used regardless of what type of collecting structure the collecting section 16 contains. A suitable material for such a shield 44 is an anionic copolymer of methacrylic acid and methyl methacrylate similar to Eugradit L100.

In one embodiment, the tentacles 42 comprise a shape-shifting material that changes shape in response to changes in temperature. A suitable choice of temperature-responsive material is Poly(N-isopropylacrylamide). Such a material remains hydrophilic when below its critical temperature but transitions into a hydrophobic state past a critical temperature. As it does so, it tends to alternate between swelling and desiccation. This causes it to change shape. Such an embodiment requires a heat source. A suitable heat source is one that is powered by an external field, such as an induction heater.

Mechanisms other than increased temperature can also be used. For example, a shape-shifting material could be made to change shape in response to chemical composition of the environment, including, for example, a change in the environment's hydrogen ion concentration, a change in the environment's hydroxide ion concentration, or a change in the environment's conductivity or salinity.

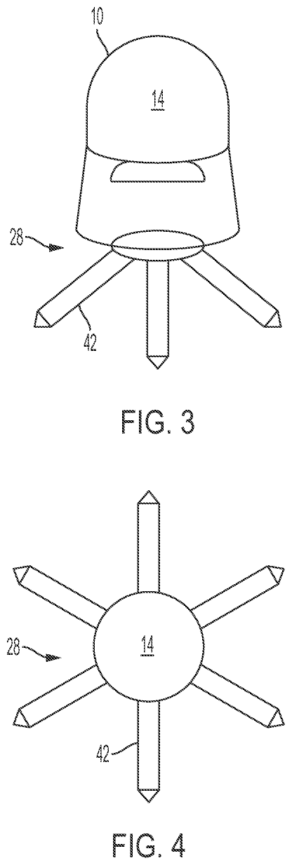

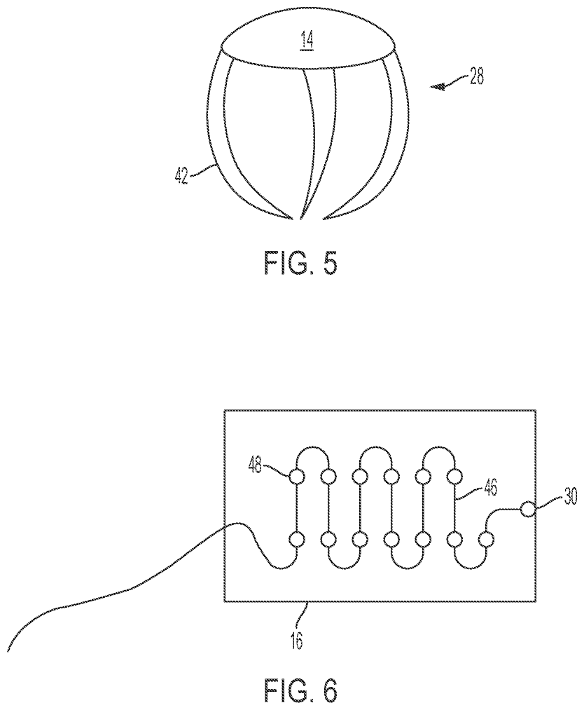

Once deployed, the tentacles 42 remain close together. But when heated, they begin to spread out as shown in FIG. 3.

In FIG. 3, the tentacles 42 are completely unfolded and ready to collect microbes. To promote its ability to collect, it is useful to coat the tentacles 42 with a material to which microbes readily adhere. In this state, the tentacles 42 harvest bacteria not only from the gut wall but also from chime and from the intestinal mucosa itself.

FIG. 4 shows the tentacles 42 in their grasping state, in which the microbes have been entrapped.

Examples of suitable coatings to promote microbial adhesion to the tentacles 42 include muco-adhesives or adhesives based on PEG, decyl-PVP, or papain.

A suitable manufacturing method for making the tentacles 42 is to form a mold from PMMA for formation of the PNIPAM film and to then stir a solution of containing a temperature-sensitive monomer, a thickener, a cross-linker, a hydrophilic monomer in a solvent and exposing it to a radiation source having photons of appropriate energy for a period of time sufficient to deposit enough energy to cause cross-linking. This will result in a suitable gel after any excess solvent has been removed.

One practice features forming a star-shaped mold from PMMA for formation of the PNIPAM film and to then stir a solution of 1.00 gram of N-Isopropylacrylamide monomer (NIPAM), 0.06 grams of N,N-methylenebisacrylamide (BIS), 0.03g 2,2-Dimethoxy-2-phenylacetophenon (PI), 0.10 milliliters methacrylic acid (MAA) and 0.28 grams polyethyleneglycol (PEG4000, molecular weight=4000) in 2.5 milliliters of n-butanol at 60 C for an hour to completely dissolve the solutes. The solution is then polymerized by exposure to radiation having a suitable wavelength. One embodiment includes irradiating with ultraviolet light for about twelve minutes and then using alcohol and de-ionized water to remove the n-butanol.

An alternative manufacturing method includes preparing aqueous solution of NIPAM (10% w/v), N, N-methylene acrylamide (BIS, 0.3% w/v) as the crosslinking agent and water-soluble PI (0.5% w/v), injecting the prepared solution into a star-shape PDMS mold, and exposing it to the ultraviolet radiation for ten minutes. This results in formation of the film's first layer, after which NIPAM 10% Chitosan 2% solution is added into the PDMS mold and cross-linked with UV to form another layer.

FIG. 6 shows a collecting section 16 that includes a capillary pump having a thread 46 coupled to the inlet 30. The thread 46 has numerous nodes 48 along its length. These nodes can be implemented as knots or absorbent pads. In some embodiments, a node is an empty cavity that fills with gastric fluid being sampled.

As a result of the thread 46 having been coupled to the inlet 30, fluid from the capsule's surroundings is able to migrate along the thread 46 via capillary action.

In some embodiments, the thread is brought into contact with the gastric fluid upon occurrence of a trigger event.

A particularly useful embodiment is one in which the thread implements what amounts to a pump. This embodiment features a fluid chamber that has a first end coupled to the exterior environment and a second end that is coupled to an internal port. When sampling is desired, the thread is brought into contact with this port. When this occurs, fluid moves from the chamber and into the thread through capillary action. This fluid that is lost from the chamber then has to be replaced. As a result, a suction pressure develops that draws fluid into the chamber from the exterior.

Since the migration rate through the thread 46 via capillary action is known, it is possible to infer when a particular sample entered the inlet 30 by inspecting where it came from along the thread 46. For example, upon recovery of the gut rover 10, if the particular node 48 in which a sample was found provides a basis for estimating where along the gut 12 it was obtained.

Suitable materials for use as a thread 46 include nylon, polyester, and cotton. In general, a thread 46 made of nylon is a well-organized arrangement of nylon filaments that provide predictable flow with only a small standard deviation in weight per unit length and water content per unit length. A thread 46 made of polyester is still somewhat organized but introduce some randomness in these properties. A thread 46 made of cotton comprises a random jumble of cotton filaments, as a result of which a thread 46 made of cotton exhibits the highest standard deviation between samples for these two properties.

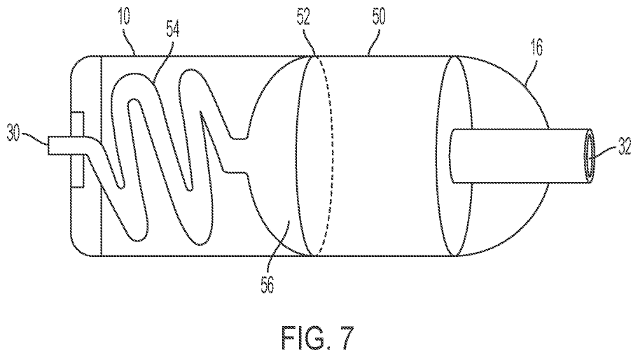

Another embodiment, which is shown in FIG. 7, relies on passive osmotic pressure. This embodiment features a brine reservoir 50 coupled to the outlet 32 and a collection channel 54 coupled to the inlet 30 with a semi-permeable membrane 52. The collection channel 54 travels along a helical path from the inlet 30 towards the brine reservoir 50.

In some embodiments, the collection channels 54 have a roughly rectangular cross section that is about 0.8 millimeters high and 2.8 millimeters wide. In a capsule with length 21 millimeters and a 7-millimeter diameter, there is room for 2.25 turns in the helix and a total sampling volume of 200 microliters.

Some embodiments feature a stilling chamber between the beginning of the collection channel 54 and outer surface of the rover 10 so that fluid from the gut passes through the inlet 30 and into the stilling chamber before entering the collection channel 54.

A first side of the semi-permeable membrane 52 faces the collection chamber 56. A second side of the semi-permeable membrane 52 faces the collection chamber 56. Gut fluid on one side of this membrane 52 flows through the semi-permeable membrane 52 in an effort to dilute the brine in the brine reservoir 50. However, microbes cannot flow through the semi-permeable membrane 52 and as a result remain trapped in the collection chamber 56.

Since the fluid continuously flows into the brine reservoir as a result of osmosis, this excess fluid must be disposed of. As a result, it is useful to provide an outlet from the brine reservoir 50 back into the gut. The diameter of this opening is important to provide sufficient flow rate to avoid having fluid enter the brine reservoir 50 from the gut. A rapid flow rate is also useful to reduce the possibility of clogging. This is particularly useful since the fluid in the gut contains a great deal of suspended particulates.

In some embodiments, the outlet has a diameter of 100 micrometers. In a typical case, this yields a fluid velocity of 0.13 millimeters per second through the outlet. In other embodiments, the outlet has a diameter of 50 micrometers. This corresponds to the resolution of a typical 3D-printer that could be used for manufacturing the rover 10. In a typical case, this yields a fluid velocity of 0.6 millimeters per second through the outlet.

A suitable semi-permeable membrane 52 is one made of cellulose acetate with a thickness of approximately three microns. Other semi-permeable membranes 52 include those made of a thin film coating of polyimide, thermoplastic polyurethane, or mixtures of cellulose acetate, ethanol, and acetone. Other suitable semi-permeable membranes 52 include reverse-osmosis membranes and nanopore membranes.

A difficulty that arises in the embodiment shown in FIG. 7 is that as water diffuses into the brine reservoir 50 under osmotic pressure, it dilutes the brine in the brine reservoir 50. At some point, it will become dilute enough to that that diffusion out of the reservoir may begin. This may result microbes within the collection channel 54 flowing back out through the inlet 30. It is therefore useful to halt the sampling process before this occurs and to trap the microbes in the collection channel 54.

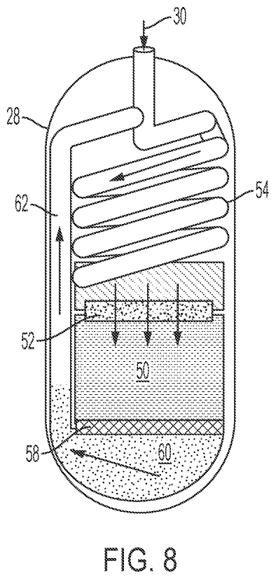

An alternative embodiment, shown in FIG. 8, features an elastic membrane 58 with a first side facing the brine reservoir 50 and a second side facing an oil reservoir 60. A suitable material from which to make the elastic membrane 58 is polydimethylsiloxane. The oil reservoir connects to a backflow channel 62 that leads to the collection channel 54 near the inlet 30.

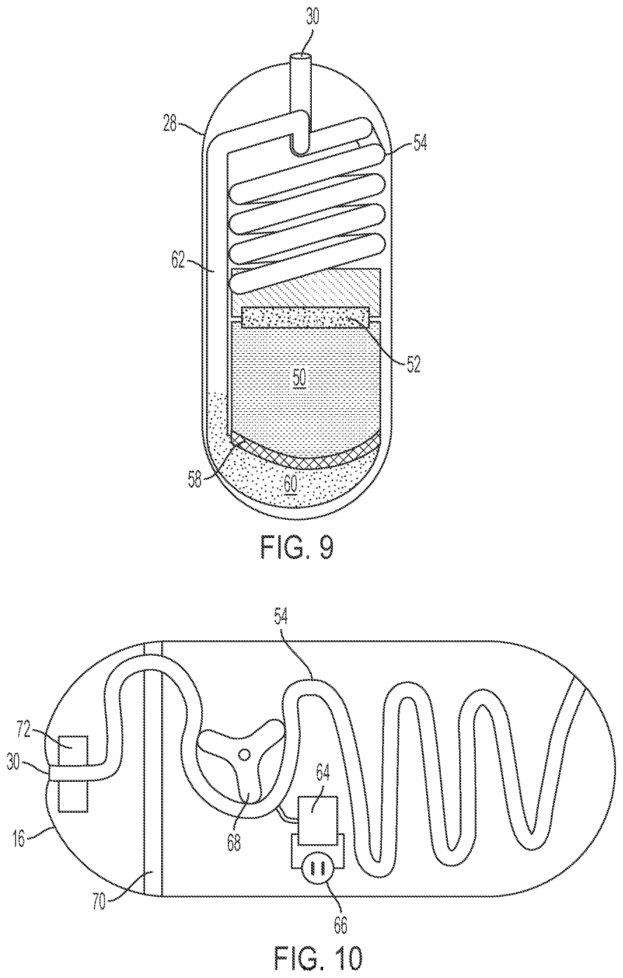

In this embodiment, the osmotic pressure deforms the elastic membrane 58 so that it bows slightly into the oil reservoir 60 thus displacing some oil. This causes the level of oil within the backflow channel 62 to rise. Eventually, the level of oil rises far enough to reach the top of the backflow channel 62, as shown in FIG. 9. At this point, oil spills into the collection channel 54 and prevents further entry of gut fluid. This halts the collection process and traps microbes in the collection channel 54.

In an alternative embodiment, shown in FIG. 10, the collecting section 16 houses a motor 64 powered by an on-board power source 66 to drive a peristaltic pump 68 that engages the collection channel 54 and thus pumps fluid from the capsule's environment through the collection channel 54. In some embodiments, an energy transducer 70 harnesses the peristaltic motion of the gut 12 itself to recharge the power source 66, thus resulting in a peristaltically-powered peristaltic pump. A suitable energy transducer 70 is one that relies on piezoelectric elements. Some embodiments harvest energy from the acidic environment of the stomach to charge or top up the power source 66 before the rover enters the intestines. A suitable power source 66 in such an application is a super capacitor.

Because the fluid in the gut 12 carries considerable quantities of particulate matter, it is particularly useful to include an anti-clogging device 72. While shown only in the embodiment of FIG. 8, an anti-clogging device 72 is useful for all embodiments of the collecting section 16 that imbibe the particulate-laden fluid that fills the gut 12. In some embodiments, an ultrasonic transducer implements the anti-clogging device 72.

In an alternative embodiment, shown in FIG. 11, the collecting section 16 houses a DC motor 64 powered by an on-board power source 66 to rotate a first pulley 74. A belt 76 extends between the first pulley 74 and a second pulley 78. The second pulley 78 lies next to the inlet 30. As a result, when the belt 76 passes over the second pulley 78, it has the opportunity to pick up microorganisms in the gut fluids. To promote the ability to do so, the belt 76 typically features a corrugated and/or sticky surface.

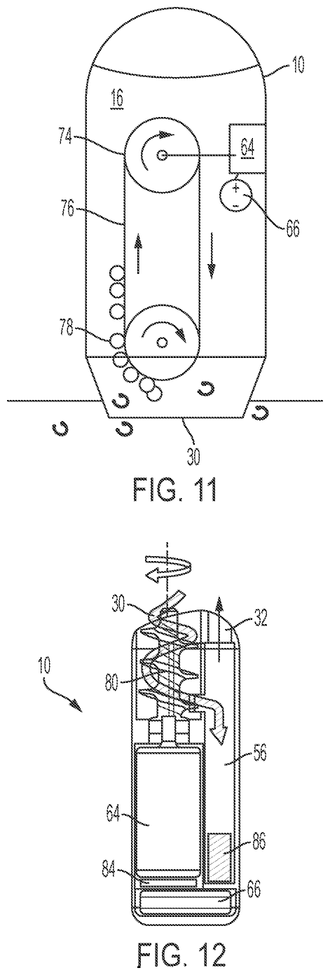

Yet another motorized embodiment, shown in FIG. 12, features a power source 66 that powers a motor 64 that rotates a screw 80 within a cylindrical cavity to draw gut fluid through the inlet 30 into the collection chamber 56 and to expel gut fluid through the outlet 32 with a collected sample 86 having been retained in the collection chamber 56. An externally-controlled switch 84 can be used to to turn the motor 52 on or off on cue to facilitate spot sampling.

In some embodiments, the switch is a magnetic reed switch that is controlled by an external magnetic field. A suitable magnetic-field source is a permanent magnet or a Helmholtz coil. In other embodiments, the switch is a transistor that can be made to transition between its conducting and non-conducting states as a result of a receiver receiving an appropriate signal from externally-generated radio waves and converting that signal, using an RF to DC converter, into a DC signal suitable for controlling the switch. A suitable receiver is one that operates in the RFISD or ISM band.

The motor 64 includes a gearbox to rotate the screw 80 at a relatively low speed, for example at between fifteen and fifty revolutions per minute. The various electrical components and the magnet are embedded in resin to avoid having their operation compromised by moisture.

Such an embodiment is particularly advantageous when the gut fluid has high viscosity or when gut fluid is so laden with particulate matter that it could more readily be characterized as semi-solid. Examples include mucus, feces, and tissue.

Having described the invention, and a preferred embodiment thereof, what is new and secured by Letters Patent is:

1. An apparatus comprising a gut rover that is configured to traverse a gut, said gut rover comprising a sampler for obtaining samples of the microbiome at selected locations within said gastrointestinal system.

2. The apparatus of claim 1, wherein said sampler comprises an osmotic sampler in which an osmotic pressure differential across a membrane drives sampling.

3. The apparatus of claim 1, wherein said sampler comprises a brine reservoir, a semi-permeable membrane, and a collection chamber that is in fluid communication with an inlet through which fluid within the gut can enter said gut rover, and wherein said semi-permeable membrane separates said brine reservoir from said collection chamber.

4. The apparatus of claim 1, wherein said sampler comprises an oil reservoir, a back channel, and an elastic membrane wherein said elastic membrane separates a brine reservoir from said oil reservoir, and wherein deformation of said elastic membrane increases a level of oil from said oil reservoir in said back channel.

5. The apparatus of claim 2, wherein said sampler is configured to halt sampling upon having collected a pre-defined volume.

6. The apparatus of claim 3, wherein said brine reservoir has a volume that expands during sampling.

7. The apparatus of claim 1, wherein said sampler comprises a thread and nodes along said thread, wherein said thread has an end exposed to fluid within said gut.

8. The apparatus of claim 1, wherein said sampler comprises a material that changes shape in response to a trigger.

9. The apparatus of claim 1, wherein said sampler comprises a material that transitions between hydrophobic and hydrophilic states in response to an energy input.

10. The apparatus of claim 1, wherein said sampler comprises tentacles that deform between an open state and a closed state, wherein in said open state said tentacles are exposed to fluid in said gut and wherein in said closed state said tentacles entrap samples from said fluid.

11. The apparatus of claim 1, wherein said sampler comprises a heater that is selectively activated by a remote trigger.

12. The apparatus of claim 1, wherein said sampler comprises a pump that is connected to an inlet of said gut rover.

13. The apparatus of claim 1, wherein said sampler comprises a peristaltic pump.

14. The apparatus of claim 1, wherein said sampler comprises a screw having threads, wherein pairs of threads confine fluid therebetween, wherein, as said screw rotates, fluid confined between pairs of threads moves along an axis of said screw.

15. The apparatus of claim 1, wherein said sampler comprises an endless belt that extends between first and second pulleys, wherein said endless belt follows a path that exposes said belt to gut fluid.

16. The apparatus of claim 1, further comprising a shield that prevents fluid from contacting said sampler, wherein said shield is configured to dissolve upon occurrence of a condition indicative of entry into region from which samples are to be acquired.

17. A method comprising providing a gut rover to a patient, after said gut rover has traversed said patient's gut, recovering said gut rover, and recovering, from said gut rover, microbes from within said gut.

18. The method of claim 17, further comprising tracking said gut rover while said gut rover is traversing said gut.

19. The method of claim 17, further comprising controlling sampling by said gut rover while said gut rover is within said gut.

20. The method of claim 19, wherein controlling sampling comprises causing a heater within said gut rover to generate heat.

21. The method of claim 19, wherein controlling sampling comprises turning on a motor within said gut rover.

22. The method of claim 18, wherein tracking said gut rover comprises observing a magnetic signature from said gut rover and identifying a location of said gut rover based on said magnetic signature.

23. The method of claim 17, further comprising reorienting said gut rover while said gut rover is within said gut.

24. The method of claim 23, wherein reorienting said gut rover comprises exposing said gut rover to a magnetic field generated outside said patient.

25. The method of claim 17, further comprising causing said gut rover to move while said gut rover is within said gut.

26. The method of claim 25, further comprising causing said gut rover to move comprises exposing said gut rover to a magnetic field generated outside said patient.

27. The method of claim 17, wherein recovering, from said gut rover, microbes from within said gut comprises recovering fluid that has been trapped behind an oil plug.

28. The method of claim 17, wherein recovering, from said gut rover, microbes from within said gut comprises recovering a thread that has been impregnated with fluid that has been gathered from said gut through capillary action.

29. The method of claim 17, wherein recovering, from said gut rover, microbes from within said gut comprises recovering said microbes from a fluid having a first concentration of water, wherein said gut has fluid that has a second concentration of water, and wherein said first concentration is less than said second concentration.
